(19) United States
(12) Patent Application Publication
(10) Pub. No.: US 2022/0032083 A1
(43) Pub. Date: Feb. 3, 2022

(54) A LIGHT-BASED SYSTEM AND METHOD FOR SHAPING AND TREATING HAIR

(71) Applicant: The General Hospital Corporation, Boston, MA (US)

(72) Inventors: William G. Austen, Boston, MA (US); Robert Redmond, Boston, MA (US); Michael McCormack, Boston, MA (US)

(21) Appl. No.: 16/624,863

(22) PCT Filed: Jun. 22, 2018
(86) PCT No.: PCT/US2018/039134

§ 371 Date: Dec. 19, 2019

Related U.S. Application Data
(60) Provisional application No. 62/523,734, filed on Jun. 22, 2017.
(60) Provisional application No. 62/523,994, filed on Jun. 23, 2017.
(60) Provisional application No. 62/558,016, filed on Sep. 13, 2017.

(51) Int. Cl. A61N 5/06; A45D 2/00; A45D 1/04

(52) U.S. Cl. CPC A61N 5/0617; A61N 5/062; A61N 2005/0658; A45D 1/04; A61N 2005/0644; A45D 2/001

(57) ABSTRACT

System and methods for treating and shaping a keratin fiber are provided. A light-based system includes an activation system configured with one or more light source. The light source is configured to apply electromagnetic radiation at a target region including the keratin fiber. The light-based system applies the electromagnetic radiation to the target region to effectuate photocrosslinking with the assistance of a photosensitizer on the keratin fiber and transition the keratin fiber from a first physical state to a second physical state.

### CROSS-REFERENCE TO RELATED APPLICATIONS

This application is based on, claims the benefit of, and incorporates herein by reference in their entirety U.S. Provisional Patent Application Ser. No. 62/523,734, filed on Jun. 22, 2017; U.S. Provisional Patent Application Ser. No. 62/523,994, filed on Jun. 23, 2017; and U.S. Provisional Patent Application Ser. No. 62/558,016, filed on Sep. 13, 2017.

### BACKGROUND

In the field of beauty and cosmetics, consumers are constantly seeking more efficient products and more effective tools for styling their hair. Such products might allow the consumer to more easily attain a style, such as straight or curly hair, while also offering improvements in volume, elasticity, and/or hold.

Currently, many hair shaping tools and products exist on the market that can generally be separated between temporary and permanent shaping methods. Each of these methods relies on disrupting and reorganizing the intermolecular interactions that are present within hair. For example, hair typically comprises fibrous proteins, such as α-keratin fibers, configured in a coiled coil secondary structure. Intermolecular interactions (i.e. hydrogen bonding, coulombic interactions, etc.) between amino acid side chains and covalent disulfide bonds between fibers dictate the secondary structure of the proteins present in hair. A change in the macromolecular structure of hair (i.e. from curly to straight) can be achieved by disrupting these stabilizing forces.

Temporary shaping methods typically disrupt the stabilizing forces present within hair through the application of heat. Heat is often applied through an apparatus, such as a blow dryer or a flat iron, at a temperature sufficient to disrupt the intermolecular interactions between the fibrous proteins. Sufficient temperatures typically range between 200 to 500 degrees Fahrenheit. During the temporary shaping method, heat is applied to the hair while simultaneously stretching or curling the hair into a desired shape. Heat is then removed, and the hair is allowed to cool in the desired shape to allow the intermolecular interactions to reform between the fibrous proteins, thereby providing hold to the new shape. Temporary shaping methods can also include applying hair-fixture polymers to the hair, such as waxes, gels, or pomades. A drawback of the temporary shaping method is that exposure to external forces, such as wind, humidity, or contact with water cause the hair to revert back to its natural shape. Furthermore, heating at elevated temperatures may cause damage to the hair.

So-called “permanent” shaping methods typically disrupt the stabilizing forces present within hair through the use of chemical reactions. In particular, permanent shaping methods use strong reducing agents to break the strong disulfide bonds present, for example, in the α-keratin fibers, followed by a neutralization step (i.e. application of hydrogen peroxide) to reform the disulfide bonds in the desired shape. Treatments typically include applying a high pH solution containing an alkaline hydroxide, such as NaOH, to the hair to induce disulfide bond reduction. Other strong reducing agents include formaldehyde, glycolic acid, and thioglycolic acid. A drawback of the permanent shaping method is that the procedure is time-consuming and can damage the hair due to the exposure to caustic chemicals. Additionally, should the consumer wish to revert from, for example, curly hair to straight hair, the procedure must be repeated or the hair allowed to grow the replace the hair that was treated. Over-exposure to the reducing agents further deteriorates the health of the hair.

Therefore, it would be desirable to provide new systems and methods for shaping hair or providing aesthetic treatments to other parts of the body to provide new, yet flexible control to the individual. Additionally, it would be desirable to provide new systems and methods for shaping and treating hair without heat or potentially harmful chemicals.

### SUMMARY

In accordance with one aspect of the present disclosure, a method is provided for performing a cosmetic treatment. The method includes applying a photosensitizer to a keratin fiber of a subject while the keratin fiber is in a first physical state and subjecting the keratin fiber and the photosensitizer to electromagnetic radiation selected to effectuate photocrosslinking or photoactivation on the keratin fiber to cause the keratin fiber to transition to a second physical state that is different than the first physical state.

In accordance with another aspect of the disclosure, a light-based system is provided for treating and shaping a keratin fiber. The light-based system includes a fluid supply system including a vessel. The vessel is configured to contain a photosensitizer. The vessel is further in fluid communication with a fluid outlet where the fluid outlet is configured to dispense the photosensitizer at a target region that includes the keratin fiber. The light-based system includes an activation system configured with one or more light source. The light source is configured to apply electromagnetic radiation at the target region. The light-based system further includes a processor having a memory and configured to be in electrical communication with the activation system. The processor is configured to execute a store program to apply electromagnetic radiation to the target region selected to effectuate photocrosslinking on the keratin fiber such that the keratin fiber transitions from a first physical state to a second physical state.

In accordance with another aspect of the present disclosure, a method of performing a cosmetic treatment is provided. The method includes applying a photosensitizer to a keratin fiber within a subject in a first physical state. The method further includes subjecting the keratin fiber and the photosensitizer to electromagnetic radiation selected to effectuate photocrosslinking on the keratin fiber to cause the keratin fiber to transition to a second physical state that is different than the first physical state.

The foregoing and other aspects and advantages of the invention will appear from the following description. In the description, reference is made to the accompanying drawings, which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

The present invention will hereafter be described with reference to the accompanying drawings, wherein like reference numerals denote like elements.

FIG. 1 is a flowchart setting forth the steps of a method for performing a cosmetic treatment according to one aspect of the present disclosure.

FIG. 2 is a block diagram of a light-based system that is configured to implement a method for performing a cosmetic treatment in accordance with one aspect of the present disclosure.

FIG. 3 is an example cosmetic device that is configured to implement a method for performing a cosmetic treatment in accordance with one aspect of the present disclosure.

FIG. 4 is an example cosmetic device that is configured to implement a method for performing a cosmetic treatment in accordance with one aspect of the present disclosure.

FIG. 5 is an example cosmetic device that is configured to implement a method for performing a cosmetic treatment in accordance with one aspect of the present disclosure.

FIG. 6 is an example cosmetic device that is configured to implement a method for performing a cosmetic treatment in accordance with one aspect of the present disclosure.

FIG. 7 is an example cosmetic device that is configured to implement a method for performing a cosmetic treatment in accordance with one aspect of the present disclosure.

FIG. 8 is an example cosmetic device that is configured to implement a method for performing a cosmetic treatment in accordance with one aspect of the present disclosure.

FIG. 9 is a non-limiting example of an image of a subject's hair prior to performing a cosmetic treatment in accordance with one aspect of the present disclosure.

FIG. 10 is a series of images illustrating the subject's hair of FIG. 9 after the cosmetic treatment, and non-limiting examples of retention of the cosmetic treatment after exposure to multiple washes.

FIG. 11 is a non-limiting example of an image of a subject's hair prior to performing a cosmetic treatment in accordance with one aspect of the present disclosure.

FIG. 12 is an image of the subject's hair of FIG. 11 after the cosmetic treatment.

FIG. 13 is a series of images illustrating the subject's hair and non-limiting examples of retention of the cosmetic treatment after exposure to multiple washes.

### DETAILED DESCRIPTION

Described herein are methods, systems, and kits for performing a cosmetic treatment, such as treating or shaping keratin fibers on a subject. Exemplary keratin fibers may be, for example, keratin fibers found in a subject's hair or nail. In general, the methods described herein include applying, or otherwise administering, a photosensitizer to a keratin fiber of a subject while the keratin fiber is in a first physical state, and subjecting the keratin fiber to electromagnetic radiation at an appropriate wavelength, energy, and duration to cause the keratin fiber to transition to a second physical state that is different than the first physical state. As used herein, the term “physical state” may refer to any measurable property of the keratin fiber within the subject. Exemplary physical states of the keratin fiber may include, but are not limited to a mass, length, density, thickness, spatial orientation, volume, tensile strength, and/or color of the subject's hair or nail.

As used herein, the term “shaping” may relate to performing a cosmetic treatment on a keratin fiber to, for example, change the physical appearance of the subject's hair or nail. Shaping the keratin fibers may include applying, or otherwise administering, a photosensitizer to a keratin fiber in a region of interest of the subject, and irradiating the keratin fiber with electromagnetic radiation at an appropriate wavelength, energy, and duration to effectuate photocrosslinking on the keratin fiber, thereby transforming the keratin fiber from a first physical state to a second physical state. In some aspects of the disclosure, a cosmetic device (e.g., brush, hair drier, curling iron, straightening iron, nail applicator) is used to assist in physically transforming the keratin fiber from the first physical state to the second physical state. In one aspect of the disclosure, altering the keratin fiber from the first physical state to the second physical state includes altering the spatial orientation of the keratin fiber and photocrosslinking the keratin fiber while it is in the second physical state such that it retains the spatial orientation of the second physical state for a period of time. Altering the spatial orientation of the keratin fiber may include changing a subject's hair from straight to curly, curly to straight, and spatial orientations therebetween.

As used herein, the term “retain” generally refers to maintaining the second physical state (e.g., curly or straight) of the keratin fibers for a period of time. Such period of time may be one appropriate for hair applications, such as at least a few hours or extending indefinitely, until the keratin fibers are arranged in a different physical state. For example, the period of time may be measured based on a number of washes, where the second physical state of the keratin fibers may be substantially retained after being submerged and agitated (e.g., scrubbed) in water or soapy water. In some aspects of the disclosure, at least a portion of the keratin fibers can retain the second physical state over a period of time where at least 10% of the second physical state is retained after a number of washes, or at least 20% of the second physical state, or at least 30% of the second physical state, or at least 40% of the second physical state, or at least 50% of the second physical state, or at least 60% of the second physical state, or at least 70% of the second physical state, or at least 80% of the second physical state, or at least 90% of the second physical state, or at least 95% of the second physical state, or more is retained after exposure to a number of washes, such as 1 wash, or 2 washes, or 3 washes, or 4 washes, or 5 washes, or 6 washes, or 7 washes, or 8 washes, or 9 washes, or 10 washes, or more.

As one non-limiting, illustrative example, the relative retention of the spatial orientation of the second physical state (e.g., curly or straight hair) after a number of washes may be monitored using the following procedure. First, a strand of hair in the second physical state may be extracted from the region of interest. One end of the strand of hair may then be fixed to a location, and the strand of hair may then be stretched until it is substantially linear. The length displacement between the resting state and stretched state is recorded. The strand of hair is then washed in water or soapy water, dried, and the measurement is repeated. The relative retention of the second physical state may then be recorded by comparing the displacement measurement before washing to the displacement measurements after washing.

In some aspects of the disclosure, altering the keratin fiber from the first physical state to the second physical state may include increasing the mass, length, density, thickness, volume, tensile strength, and/or color of the subject's hair or nail. In some forms, an exogenous fiber (e.g., fibrous protein or synthetic fiber) may be added to the region of interest prior to altering the keratin fiber from the first physical state to the second physical state. For example, prior to applying electromagnetic radiation to the target region, the exogenous fiber can be added to the photosensitizer solution and the keratin fiber in the target region. Alternatively or additionally, the exogenous fiber may be applied as a solid, for example, a powder or in the form of hair extensions. Electromagnetic radiation can then be applied to the target region to effectuate photocrosslinking between the fibrous protein and the keratin fiber in the region of interest such that the keratin fiber transforms from a first physical state to a second physical state. The second physical state of the keratin fiber may have a mass, mean diameter, volume, density, tensile strength, or length that is greater than the first physical state of the keratin fiber. In other aspects, the second physical state of the keratin fiber may comprise a color that is different than the color of the first physical state.

A suitable exogenous fibrous protein may comprise keratin, collagen, synthetic fibers, artificial hair, hair, derivatives and mixtures thereof. In one non-limiting example the synthetic fibers comprise vinyl chloride, modacrylic, vinylidene chloride, polyester, nylon, derivatives and mixtures thereof. The exogenous fibrous protein or synthetic fiber may be in the form of powders or hair extensions. Exemplary uses of the cosmetic treatment may include photocrosslinking the fibrous protein to a subject's nail to increase the density of the nail. Similarly, the fibrous protein may be photocrosslinked to the subject's hair to thicken thinning regions or to increase the length (i.e. attach a hair extension). Previous hair extension methods require keratin-based glues, heat, or ultrasonic waves to implement the extension. These previous methods further require long hours (i.e. 3-4 hours) and extensive maintenance. Unlike previous methods, the present disclosure provides a safer and faster application time that be performed without the addition of heat or potentially harmful chemicals. For example, the fibrous protein may be photocrosslinked to the keratin fiber in the target region in an application time of about 30 minutes, or 15 minutes, or 10 minutes, or 5 minutes, or 2 minutes, or less than 1 minute. In some aspects of the disclosure, the exogenous fiber may be suspended, or otherwise dissolved, in a biocompatible buffer or solution at a concentration of 0.01% (w/w) exogenous fiber to solution, or 0.05% (w/w), or 0.1% (w/w), or 0.5% (w/w), or 1% (w/w), or 2% (w/w), or 3% (w/w), or 4% (w/w), or 5% (w/w), or 6% (w/w), or 7% (w/w), or 8% (w/w), or 9% (w/w), or 10% (w/w), or more.

The fibrous protein may comprise a color such as blonde, brunette, brown, black, red, gray, platinum, or the like. The fibrous protein may be photocrosslinked to the keratin fiber in the target region to alter the color of the target region.

In some aspects of the disclosure, the retention of the second physical state relative to the first physical state can be measured explanting a keratin fiber from the region of interest while the keratin fibers are in the first physical state and performing a measurement, explanting a keratin fiber from the region of interest while the keratin fibers are in the second physical state and performing a measurement, and monitoring the changes in the measurements between the first physical state and the second physical state. In some aspects of the disclosure, at least a portion of the keratin fibers can retain the second physical state over a period of time where at least 10% of the second physical state is retained relative to the first physical state, or at least 20% of the second physical state, or at least 30% of the second physical state, or at least 40% of the second physical state, or at least 50% of the second physical state, or at least 60% of the second physical state, or at least 70% of the second physical state, or at least 80% of the second physical state, or at least 90% of the second physical state, or at least 95% of the second physical state, or more is retained relative to the first physical state for a period of time, for example, for 1 day, or 1 week, or 2 weeks, or 3 weeks, or 4 weeks, or 1 month, or 2 months, or 3 months, or 4 months, or 5 months, or 6 months, or 7 months, or 8 months, or 9 months, or 10 months, or 11 months, or 1 year, or more. Suitable measurements to monitor the changes in the physical state from the first physical state to the second physical state include weight measurements, volume displacement measurements, colorimetry measurements, length measurements, and/or tensile strength measurements of the keratin fiber.

Alternatively or additionally, the physical state of the keratin fiber may also relate to an identifiable condition of a disease, or a concentration of a chemical species indicative of the disease. For example, nail diseases (e.g., onychosis) may be identified based on signs of infection or inflammation. Exemplary physical states may also include, but are not limited to, discoloration of the nail (e.g., yellowing, browning, and redness), shape and texture of the nail (degree of nail clubbing, koilonychias, pitting due to psoriasis, beau's lines), and pliability of nail (brittleness measured by iron concentration, splitting and fraying associated with folic acid, protein, and vitamin C deficiencies).

As used herein, the term “treating” may relate performing a cosmetic treatment to induce therapeutic changes. In some aspects of the disclosure, a therapeutically effective amount or pharmaceutically appropriate dosage of a photosensitizer is applied to a keratin fiber in the region of interest, and the keratin fiber is irradiated with electromagnetic radiation at an appropriate wavelength, duration, and intensity to elicit a biological or medical response of a subject, tissue, or cell that is being sought by the researcher, veterinarian, medical doctor, or other clinician. In some aspects of the disclosure, the biological or medical response is elicited by photoactivating the photosensitizer. In some aspects of the disclosure, the altering the keratin fiber from the first physical state to the second physical state may include reducing the degree or intensity of the identifiable condition of a disease or the concentration of a chemical species indicative of the disease, for example reducing the amount of fungus within the subject's nail, or reducing the amount of lice in the subject's hair. The term “subject” as used herein may refer to both human subjects and other animal subjects including domestic large and small animals such as dogs, cats, rabbits, horses, cows, pigs, and the like.

As used herein, “photoactivation” is used to describe the process by which energy in the form of electromagnetic radiation is absorbed by a compound, such as a photosensitizer. The electromagnetic radiation can include energy, e.g., light, having a wavelength in the visible range or portion of the electromagnetic spectrum, or the ultra violet and infrared regions of the spectrum. The chemical energy can be in the form of reactive species, such as a singlet oxygen, superoxide anion, hydroxyl radical, the excited state of the photosensitizer, photosensitizer free radical, or substrate free radical species. The photoactivation process described herein may involve insubstantial transfer of the absorbed energy into heat energy. Preferably, photoactivation occurs with a rise in temperature of less than 15 degrees Celsius, or a rise of less than 10 degrees Celsius, or a rise of less than 3 degrees Celsius, or a rise of less than 2 degrees Celsius, or a rise in temperature of less than 1 degree Celsius as measured, e.g., by an imaging thermal camera that looks at the target region during irradiation. The camera can be focused in the area of original dye deposit, e.g., target region comprising a keratin fiber, or on an area immediately adjacent the target region, to which dye will diffuse.

In some aspects of the disclosure, the term “photosensitizer” includes a chemical moiety that absorbs electromagnetic radiation, by a process such as photoactivation, to effectuate the formation of a covalent bond between a keratin fiber and another macromolecule or between two different parts of an individual keratin fiber.

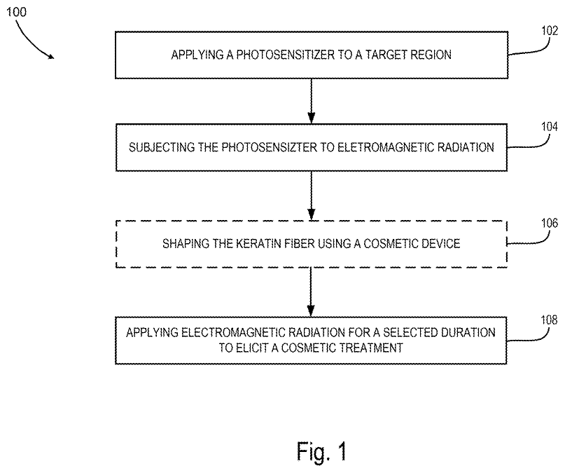

Referring particularly now to FIG. 1, an example method of performing a cosmetic treatment 100 is shown. The method includes applying, or otherwise administering, a photosensitizer 102 to a keratin fiber of a subject. The photosensitizer may be applied 102 manually, for example, using a spray bottle. Alternatively or additionally, the photosensitizer may also be applied 102 using a fluid dispensing system, for example, using a pump, nozzle, fluid supply tank, and a control unit to regulate the flow and dispensing rate. The photosensitizer may be dissolved in a biocompatible buffer or solution, e.g., saline solution, and used at a concentration of from 0.1 mM to 10 mM, or from 0.5 mM to 5 mM, or from 1 mM to 3 mM. Alternatively, the photosensitizer may be dissolved or suspended within a gel, such as a hydrogel or a silicone gel. The keratin fiber may be applied on an external surface or an internal surface of the subject. An amount of photosensitizer may be applied that is sufficient to cover at least a portion of the keratin fiber. For example, at least 10 μL of photosensitizer solution, or greater than 50 μL, 100 μL, 250 μL, 500 μL, or 1 ml, or more can be applied to the target region. In some aspects, the photosensitizer may be dissolved in the biocompatible buffer or solution at a concentration of 0.01% (w/w) photosensitizer to solution, or 0.05% (w/w), or 0.1% (w/w), or 0.5% (w/w), or 1% (w/w), or 2% (w/w), or 3% (w/w), or 4% (w/w), or 5% (w/w), or 6% (w/w), or 7% (w/w), or 8% (w/w), or 9% (w/w), or 10% (w/w), or more.

Next, electromagnetic radiation may be applied 104 to the keratin fiber and the photosensitizer. The electromagnetic radiation may be applied 104 at an appropriate wavelength, energy, and duration, to cause the photosensitizer to effectuate photocrosslinking on the keratin fiber, thereby transforming the keratin fiber from a first physical state to a second physical state. In some aspects of the disclosure, the electromagnetic radiation may be applied 104 at a therapeutically effective amount, or a pharmaceutically appropriate dosage, to elicit a biological or medical response to treat a disease or condition in the region of interest, thereby transforming the keratin fiber from a first physical state to a second physical state. In some aspects, a cosmetic device may be used to assist in shaping the keratin fiber 206 from the first physical state to the second physical state, such as by applying a force or/and or changes in temperature. This may involve, for example, shaping the hair using a brush or a curling iron prior to or during the application of electromagnetic radiation to the keratin fiber. The electromagnetic radiation may be applied for a selected duration 108 to elicit a response, such as to effectuate photocrosslinking on the keratin fiber or to induce photoactivation to treat a disease or condition. For example, the duration of irradiation can be from about 1 second to 30 minutes. In other aspects, the duration of irradiation ranges from about 1 second to 30 seconds, or 30 seconds to 2 minutes, or 2 minutes to 5 minutes, or greater than 5 minutes. The wavelength of light can be chosen so that it corresponds to the absorption of the photosensitizer, and reaches the area of the keratin fiber that has been contacted with the photosensitizer, e.g., penetrates into the region where the photosensitizer presents. The light source may be configured to apply electromagnetic radiation at a radiant energy that is less than 2000 J/cm², or between 50 and 200 J/cm². In some aspects, the light source may be configured to apply electromagnetic radiation at a radiant energy between 60 to 120 J/cm², or 80 to 100 J/cm². The electromagnetic radiation necessary to achieve photoactivation of the photosensitizer agent can have a wavelength from about 350 nm to about 800 nm, or from about 400 to 700 nm and can be within the visible, infrared or near ultraviolet spectra. The energy can be delivered at an irradiance of about between 0.1 and 5 W/cm², or between about 0.5 and 2 W/cm².

In some aspects, the second physical state may be tunable to range from a temporary transformation, a semi-permanent transformation, or a permanent transformation. In the context of a hair strand, determining whether a transformation is temporary or permanent may be measured as the hair's resistance to change after being exposed to moisture, such as a number of washes using water. A temporary transformation may be defined as a keratin fiber that reverts from the second physical state to the first physical state after exposure to 1 wash. A permanent transformation may be defined as a keratin fiber that that does not revert from the second physical state to the first physical state after exposure to 10 washes. A semi-permanent transformation falls within these bounds. The state of the transformation may be tuned, for example, by altering the wavelength, energy, or duration of electromagnetic radiation applied to the target region. Alternatively, the state of the transformation may be tuned by changing the concentration of the photosensitizer in the target region. In some aspects, the light-based system 100 may effectuate photocrosslinking in a keratin fiber such that the second physical state of the keratin fiber is resistant to at least 1 wash, at least 2 washes, at least 3 washes, at least 4 washes, at least 5 washes, at least 6 washes, at least 7 washes, at least 8 washes, at least 9 washes, or at least 10 washes.

In one non-limiting example method, a gel comprising the photosensitizer can be applied or administered to a subject's eyelid. A cosmetic device, such as a mascara brush, may then be used to brush a subject's eyelashes into the gel to place the keratin fibers positioned within the subject's eyelashes in contact with the photosensitizer. Alternatively, the gel may be applied or administered without the photosensitizer, and the photosensitizer may be applied manually to the subject's eyelashes by any of the methods described above. The subject's eyelashes may be configured to stick to the gel, or the eyelashes may be pulled away for further treatment prior to contacting the keratin fibers with the photosensitizer. A light source may then apply electromagnetic radiation to the subject's eyelashes to effectuate photocrosslinking on the keratin fibers, thereby transforming the keratin fiber from a first physical state to a second physical state. A protective cover can be used during treatment to block the electromagnetic radiation from entering the eye. In another non-limiting example, the cosmetic device may be used to brush the gel comprising the photosensitizer into the subject's eyelashes. The subject's eyelashes may then be stuck to the subject's eyelid or pulled away prior to treatment. Suitable gels include hydrogels, silicone gels, or the like. In some aspects, the gel may function as an optical wave guide to assist in delivering the electromagnetic radiation to the keratin fibers. For example, the silicone gel may comprise a reflective base that reflects the electromagnetic radiation and guides the electromagnetic radiation to the keratin fibers.

Suitable photosensitizers include, but are not limited to, a fluorescein, such as Rose Bengal (RB) (e.g., 4,5,6,7-tetrachloro-2′,4′,5′,7′-tetraiodofluorescein or derivatives thereof); riboflavin (e.g., 7, 8-Dimethyl-10-[(2S,3S,4R)-2,3,4,5-tetrahydroxypentyl]benzo[g]pteridine-2,4-dione or derivatives thereof); riboflavin-S-phosphate (R-5-P); methylene blue (MB); and N-hydroxypyridine-2-(1H)-thione (N-HTP), derivatives and mixtures thereof.

Other examples of photosensitive compounds that may be used in the fluid supply system 104 include various light-sensitive dyes and biological molecules such as, for example, Photofrin®, synthetic diporphyrins and dichlorins, phthalocyanines with or without metal substituents, chloroaluminum phthalocyanine with or without varying substituents, O-substituted tetraphenyl porphyrins, 3,1-meso tetrakis (o-propionamido phenyl) porphyrin, verdins, purpurins, tin and zinc derivatives of octaethylpurpurin, etiopurpurin, hydroporphyrins, bacteriochlorins of the tetra(hydroxyphenyl) porphyrin series (e.g., protoporphyrin I through protoporphyrin IX, coproporphyrins, uroporphyrins, mesoporphyrins, hematoporphyrins and sapphyrins), chlorins, chlorin e6, mono-1-aspartyl derivative of chlorin e6, di-1-aspartyl derivative of chlorin e6, tin(IV) chlorin e6, meta-tetrahydroxphenylchlorin, benzoporphyrin derivatives, benzoporphyrin monoacid derivatives, tetracyanoethylene adducts of benzoporphyrin, dimethyl acetylenedicarboxylate adducts of benzoporphyrin, Diels-Adler adducts, monoacid ring “a” derivative of benzoporphyrin, sulfonated aluminum PC, sulfonated AlPc, disulfonated, tetrasulfonated derivative, sulfonated aluminum naphthalocyanines, naphthalocyanines with or without metal substituents and with or without varying substituents, chlorophylis, bacteriochlorophyll A, anthracenediones, anthrapyrazoles, aminoanthraquinone, phenoxazine dyes, thiazines, methylene blue, phenothiazine derivatives, chalcogenapyrylium dyes, cationic selena and tellurapyrylium derivatives, ring-substituted cationic PC, pheophorbide derivative, naturally occurring porphyrins, hematoporphyrin, ALA-induced protoporphyrin IX, endogenous metabolic precursors, 5-aminolevulinic acid, benzonaphthoporphyrazines, cationic imminium salts, tetracyclines, lutetium texaphyrin, texaphyrin, tin-etio-purpurin, porphycenes, benzophenothiazinium, xanthenes, rose bengal, eosin, erythrosin, cyanines, merocyanine 540, selenium substitued cyanines, flavins, riboflavin, proflavin, quinones, anthraquinones, benzoquinones, naphthaldiimides, naphthalimides, victoria blue, toluidine blue, dianthroquinones (e.g., hypericin), fullerenes, rhodamines and photosensitive derivatives thereof.

A suitable fibrous protein may comprise keratin, collagen, synthetic fibers, artificial hair, hair, and the like. In one non-limiting example the synthetic fibers comprise vinyl chloride, modacrylic, vinylidene chloride, polyester, nylon and mixtures thereof. Therapeutics may be coupled onto the fibrous protein prior to application to the target region. Therapeutics may include small molecule drugs, nucleic acid constructs (i.e. siRNA, aptamers, ribozymes, antisense oligonucleotides, and the like), and antibody constructs. In one non-limiting example, an antifungal medication may be coupled to the fibrous protein to provide a therapeutic effect. Example antifungal medications include clotrimazole, econazole, ketoconazole, miconazole, tioconazole, terbinafine, amorolfine, and the like.

A Light-Based System:

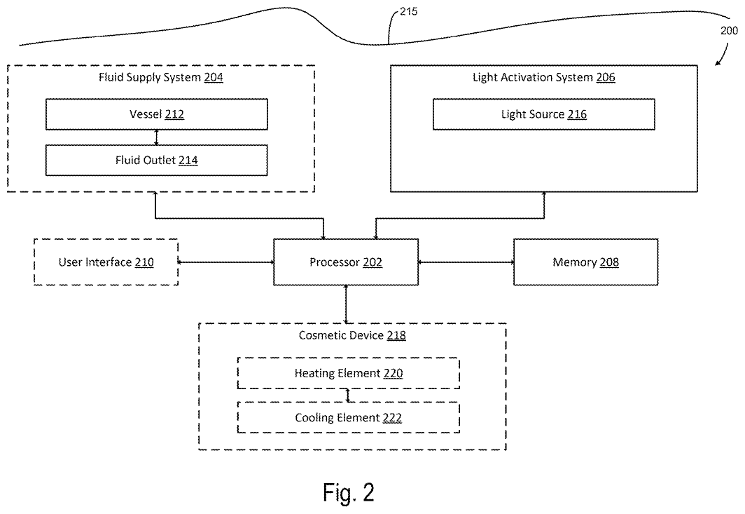

Referring particularly now to FIG. 2, an example of a light-based system 200 is illustrated that may perform the methods presented herein. In general, the light-based system 200 includes a processor 202 that is configured to be in electrical communication with a variety of components. The processor 202 may communicate with a pump or other components of a fluid supply system 204 (optional) and a light activation system 206. The optional fluid supply system 204 and the light activation system 206 or components thereof may be connected to the processor 202 by any suitable network connection, whether wired, wireless, or a combination of both. The processor 202 includes a commercially available programmable machine running on a commercially available operating system. That is, the processor 202 is configured with a memory 208 having stored programmable instructions therein. The processor 202 is capable of communicating with the fluid supply system 204 and the light activation system 206, processing data based on programmable instructions stored in the memory 208, and generating instructions. The processor 202 may be coupled to a user interface 210 that allows input parameters to be entered into the light-based system 200. The user interface 210 may be a switch or button or collection of switches or buttons. Also, the user interface 210 may include other interface components, such as displays or touch screens. To that end, the user interface 210 may also display the results.

The fluid supply system 204 may include a vessel 212 in fluid communication with a fluid outlet 214. The fluid supply system 204 is configured to dispense a fluid comprising a photosensitizer through the fluid outlet 214 towards a target region on a subject. For example, the target region can include a keratin fiber 215 on the subject, such as a strand of hair or nail. In one aspect, the optional fluid supply system 204 functions in response to instructions from the processor 202 to dispense the fluid from the fluid outlet 212 to the target region on the subject. The processor 202 may regulate the flow rate of the fluid by communicating with a pump or valve within the fluid supply system 204. In some aspects, the fluid outlet 212 is coupled to a nozzle to dispense the fluid in a spray pattern, such as a conical mist, flat fan, or a steady stream. Alternatively, instead of the fluid supply system 104, a user can employ manual dispensing systems, such as a spray bottle, syringe, or pressurized vessel.

The light activation system 206 functions in response to instructions from the processor 202 to operate a light source 216 configured to apply electromagnetic radiation to the target region on the subject. Suitable light sources 216 include commercially available lasers, lamps, light emitting diodes, or other sources of electromagnetic radiation. Light radiation can be supplied in the form of a monochromatic laser beam, e.g., an argon laser beam or diode-pumped solid state laser beam. Light can also be supplied to a non-external surface tissue through an optical fiber device, e.g., the light can be delivered by optical fibers threaded through a small gauge hypodermic needle or an arthroscope. Light can also be transmitted by percutaneous instrumentation using optical fibers or cannulated waveguides.

In some aspects, the light-based system 100 includes a cosmetic device 118. The cosmetic device 118 is configured to assist in altering the keratin fiber from a first physical state to a second physical state such as by applying a force to the keratin fiber. The cosmetic device 118 may include a heating element 120 to assist in disrupting the non-covalent interactions within the keratin fiber, or a cooling element 122 to assist in reforming the non-covalent interactions. For example, if the keratin fiber is positioned within a strand of hair of the subject, the cosmetic device 118 may include a curling iron, a hair straightener, a blow dryer, a mascara brush, a hair brush, and the like. In the instance the keratin fiber is positioned within a nail of the subject the cosmetic device 118 may include a nail polish brush, sponge applicator, and the like.

In one aspect, the light-based system 100 may be useful in shaping a subject's hair to retain a particular physical appearance that is resistant to moisture and multiple washes. For example, the light-based system 100 can be used to alter a keratin fiber positioned within a subject's hair to change from a first physical state, e.g. straight hair, to a second physical state, e.g. curly hair, or vice versa. As described above, cosmetic devices 118 may be used to assist in shaping the hair prior to photocrosslinking.

Photocrosslinkng offers several benefits over conventional methods for shaping hair. First, the methods described herein may be performed using no heat or at a sufficiently low temperature to avoid heat induced damage to the hair. Second, unlike conventional hair shaping methods that rely on disrupting and reforming non-covalent interactions, the present disclosure forms strong covalent bonds between and within the keratin fibers that result in improved hair hold and resistance to external forces.

Cosmetic Devices:

Various cosmetic devices 118 may be used with the light-based system 100 to assist in shaping or treating the keratin fiber. In some aspects of the disclosure, the cosmetic devices 118 may be configured to implement the methods described herein. The various devices are described below.

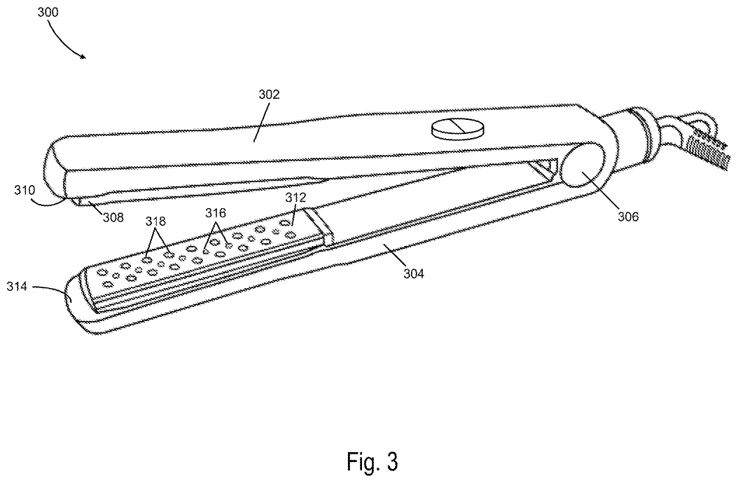

Referring to FIG. 3 an example of a cosmetic device 300 is illustrated that may be configured to implement the methods presented herein. In general, the cosmetic device 300 includes a first handle 302 and a second handle 304 extending from a hinge 306. The first handle 302 includes a first conductive element 308 attached on a first inner surface 310 of the first handle 302. The second handle 304 includes a second conductive element 312 attached on a second inner surface 314 of the second handle 304. At least one heating element may be placed in contact with the first conductive element 308 and the second conductive element 312. The cosmetic device 300 may include at least one fluid dispensing port 316 that is placed in fluid communication with the fluid supply system 204. In some aspects of the disclosure, the fluid supply system 204 may be configured within the cosmetic device 300. For example, the fluid supply system 204 may be configured within the first handle 302 or the second handle 304. Alternatively, the fluid supply system 204 may be configured external to the cosmetic device 300 and a conduit may place the dispensing port 316 in fluid communication with the fluid supply system 204. The dispensing port 316 may be configured to dispense the photosensitizer to a target region. In one aspect the target region is positioned between the first conductive element 308 and the second conductive element 312.

The cosmetic device 300 includes at least one light dispensing port 318 configured to the activation system 106. The light source 116 of the activation system 106 may be configured within the at least one light dispensing port 318 to apply electromagnetic radiation at the target region. The processor 202 may be placed in electrical communication with the at least one heating element to control the temperature of the first conductive element 308 and the second conductive element 312. In some aspects of the disclosure, the processor 202 is configured within the cosmetic device 300.

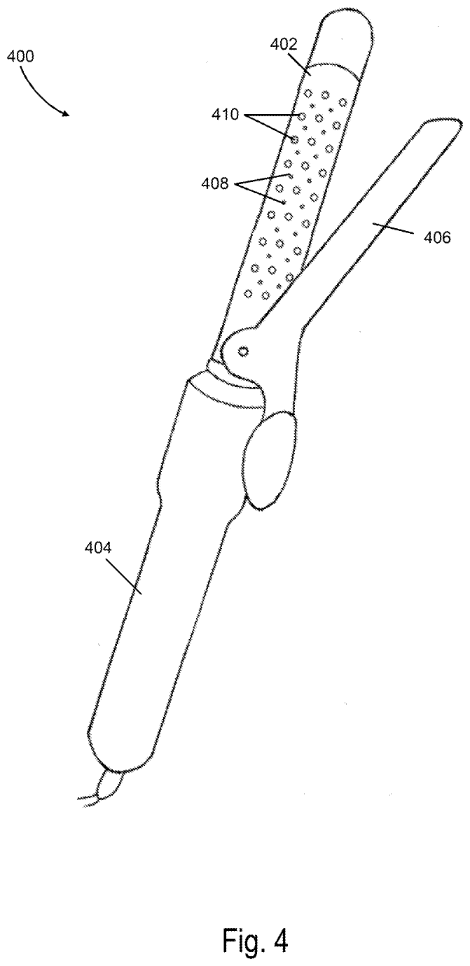

Referring to FIG. 4 an example of a cosmetic device 400 is illustrated that may be configured to implement the methods presented herein. The cosmetic device 400 includes a heat conducting body 402 having an exterior surface and a hollow center. The heat conducting body 402 may include at least one heating element disposed within the hollow center to heat the heat conducting body 402. A handle 404 is configured to the heat conducting body, wherein the handle 404 is composed of a material that does not substantially conduct heat. The cosmetic device 400 further includes a clamp member 406 pivotally connected to the heat conducting body 402. The clamp member 406 may be moved between an open position and a closed position, where the clamp member is configured to rest on the exterior surface of the heat conducting body 402 when the clamp member 406 is in the closed position. The cosmetic device includes at least one fluid dispensing port 408 that is placed in fluid communication with the fluid supply system 204. For example, the fluid supply system 204 may be configured within the handle 404. Alternatively, the fluid supply system 204 may be configured external to the cosmetic device 400 and a conduit may place the dispensing port 416 in fluid communication with the fluid outlet 214 of the fluid supply system 204. The dispensing port 416 may be configured to dispense the photosensitizer to a target region. In one aspect the target region is positioned between the heat conducting body 402 and the clamp member 406.

The cosmetic device 400 includes at least one light dispensing port 410 configured to the activation system 206. The light source 216 of the activation system 206 may be configured within the at least one light dispensing port 410 to apply electromagnetic radiation at the target region. The processor 202 may be placed in electrical communication with the at least one heating element to control the temperature of the heat conducting body 402. In some aspects of the disclosure, the processor 202 is configured within the cosmetic device 400.

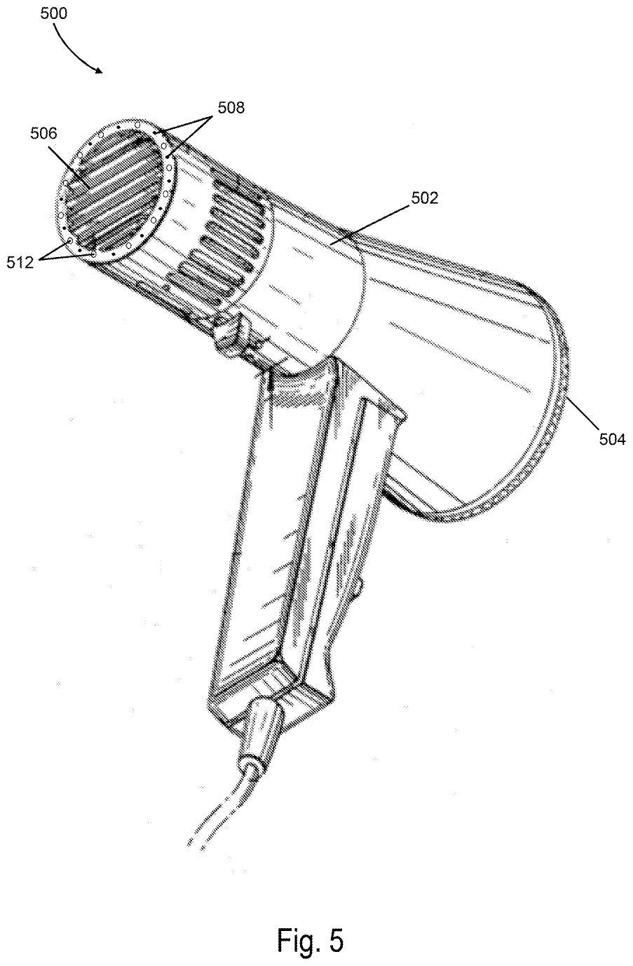

Referring to FIG. 5 an example of a cosmetic device 500 is illustrated that may be configured to implement the methods described herein. The cosmetic device 500 includes an elongated housing 502 extending from an inlet 504 to an outlet 506 to define an airflow axis. The cosmetic device 500 includes a fan positioned within the elongated housing 502 where the fan is configured to draw air into the elongated housing 502 through the inlet 504 and force air through the outlet 506 to a target region. In some aspects, the target region is defined along the airflow axis. The elongated housing 502 is configured with at least one heating element disposed within the housing. The cosmetic device 500 includes at least one fluid dispensing port 508 configured to the cosmetic device includes at least one fluid dispensing port 508 that is placed in fluid communication with the fluid supply system 204. For example, the fluid supply system 204 may be configured within a handle 510 configured to the elongated housing 502. Alternatively, the fluid supply system 204 may be configured external to the cosmetic device 500 and a conduit may place the dispensing port 508 in fluid communication with the fluid outlet 214 of the fluid supply system 204. The dispensing port 506 may be configured to dispense the photosensitizer to the target region.

The cosmetic device 500 includes at least one light dispensing port 512 configured to the activation system 206. The light source 216 of the activation system 206 may be configured to the at least one light dispensing port 512 to apply electromagnetic radiation at the target region. The processor 202 may be placed in electrical communication with the at least one heating element to control the temperature of air being delivered to the target region. In some aspects of the disclosure, the processor 202 is configured within the cosmetic device 500.

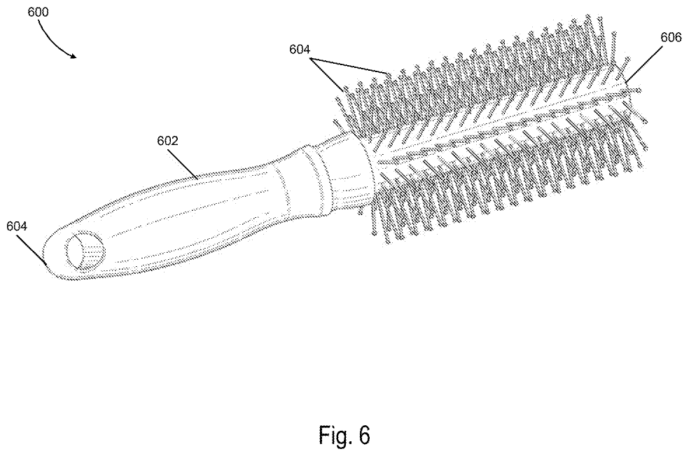

Referring to FIG. 6, an example of a cosmetic device 600 is illustrated that may be configured to implement the methods described herein. The cosmetic device 600 includes a handle 602 extending from a first end 604 to a second end 606. The handle 602 includes at least one filament 608 extending from the handle 602, for example, perpendicularly from an exterior surface of the handle 602. The at least one filament 608 is configured to the light source 216 of the activation system 206 to apply electromagnetic radiation at a target region. For example, the at least one filament 608 may be include an optical fiber or optical cable that may transmit electromagnetic radiation to the target region. In other aspects, the filament may comprise a light translucent material that transmits electromagnetic radiation to the target region. The activation system 106 may be configured within a hollow chamber positioned within the handle 602. In some aspects of the disclosure the at least one filament 608 may be coated with a photosensitizer, which may be in the form of a gel. The photosensitizer may then be transferred to the target region though the at least one filament 608. The cosmetic device 600 may include at least one fluid dispensing port configured to the cosmetic device 600 (e.g., in the at least one filament 608) includes at least one fluid dispensing port that is placed in fluid communication with the fluid supply system 204. For example, the fluid supply system 204 may be configured within the handle 602. Alternatively, the fluid supply system 204 may be configured external to the cosmetic device 600 and a conduit may place the dispensing port in fluid communication with the fluid outlet 214 of the fluid supply system 204. The dispensing port may be configured to dispense the photosensitizer to the target region.

In one aspect, the target region may be a distance from the at least one filament 604 wherein the distance is less than 1 cm, 2 cm, 3 cm, 4 cm, or 5 cm. In one aspect, the activation system 106 may be configured within a hollow center of the handle

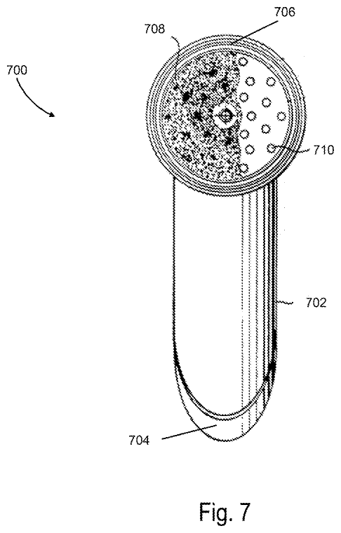

Referring to FIG. 7, an example of a cosmetic device 700 is illustrated. The cosmetic device 700 includes a handle 702 extending from a first end 704 to a second end 706. The second end 706 is configured to a porous media 708. The porous media being composed of a material that absorbs fluids, such as water or water-based solutions. For example, the porous media 708 may comprise cellulose wood fibers or foamed plastic polymers. The second end 706 is further configured with at least one light dispensing port 512 configured to the activation system 206. The light source 216 of the activation system 206 may be configured to the at least one light dispensing port 512 to apply electromagnetic radiation at the target region. The porous media 708 may be used to absorb a photosensitizer solution and apply it to a keratin fiber in a target region, such as a subject's nail.

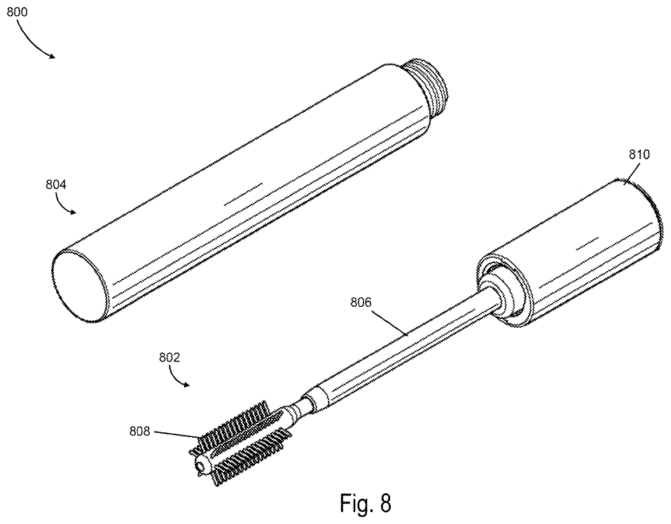

Referring to FIG. 8, an example of a cosmetic device 800 is illustrated. The cosmetic device 800 includes an applicator 802 and a container 804 that is configured to receive a photosensitizer 202. The photosensitizer 202 may be dissolved in a liquid solution, such as a saline solution. Alternatively, the photosensitizer 202 may be dissolved or suspended in a gel, such as a hydrogel or a silicone gel. The applicator 802 includes a stem 806 that extends between an applicator head 808 and a handle 810. The handle 810 is configured to be removeable coupled to a top face of the container 804, for example by a screw thread, and the stem 806 is configured to be disposed within the container 804 to place the applicator head 808 in contact with the photosensitizer 202. At least one filament 810 is configured to extend from an exterior surface of the applicator head 808. The at least one filament 810 is configured to the light source 216 of the activation system 206 to apply electromagnetic radiation at a target region. For example, the at least one filament 810 include an optical fiber or optical cable that transmits the electromagnetic radiation to the target region. In other aspects, the filament 810 may comprise a light translucent material that transmits electromagnetic radiation to the target region.

The systems and methods described herein are suitable for use in a variety of applications, including in vitro laboratory applications, ex vivo and in vivo keratin fiber treatments on living subjects. The methods are particularly useful for cosmetic treatments and applications, where keratin fibers within a target region are altered from a first physical state to a second physical state that is induced by photocrosslinking or photoactivation.

### EXAMPLES

The following examples set forth, in detail, ways in which the system may be used or implemented, and will enable one of skill in the art to more readily understand the principles thereof. The following examples are presented by way of illustration and are not meant to be limiting in any way.

### Example 1

In Example 1, a photosensitizer was used to increase the tensile strength of a subject's strand of hair. In the example, 0.1% Rose Bengal solution was applied to the strand of hair, and the hair was irradiated with green light. The load at break (Newtons) was measured for the single strain of hair with no wash and after multiple washes. The wash protocol included shampooing the hair, washing in water, conditioning the hair, and washing with water. The results are summarized below:

| | Control (N) | PXL (N) | % Diff | P Value |
|---|---|---|---|---|
| No Wash | 0.868 | 1.069 | −23% | 0.02 |
| One Wash | 0.787 | 0.969 | −23% | 0.05 |
| Two Wash | 0.753 | 0.974 | −29% | 0.01 |
| Combined | 0.803 | 1.004 | −25% | 0.0001 |
| Sample Size/Time Point | 15 | 15 | | |

### Example 2

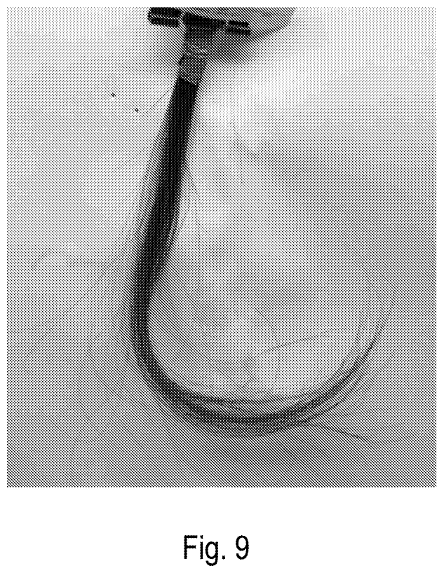

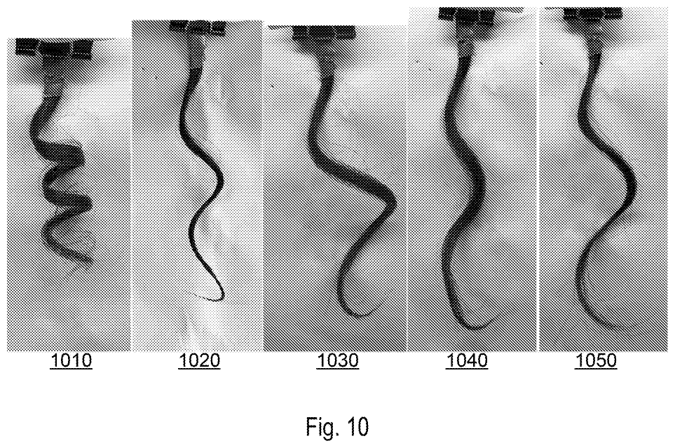

In Example 2, a solution comprising a photosensitizer and exogenous fiber was used to perform a cosmetic treatment on a subject's hair. FIG. 9 shows an image of the subject's hair prior to the cosmetic treatment. In the example, a phosphate-buffered saline (PBS) solution having a concentration of 4% (w/w) Keratin and 0.1% (w/w) Rose Bengal was applied to a subject's hair, and the hair was physically altered from its original shape into a curled state without the application of heat or other chemicals. The hair was then irradiated with electromagnetic radiation for a duration. FIG. 10 shows a series of images that illustrate the subject's hair after treatment 1010, after one wash 1020, after two washes 1030, after three washes 1040, and after four washes 1050. The washing protocol included shampooing the hair, and washing with water.

### Example 3

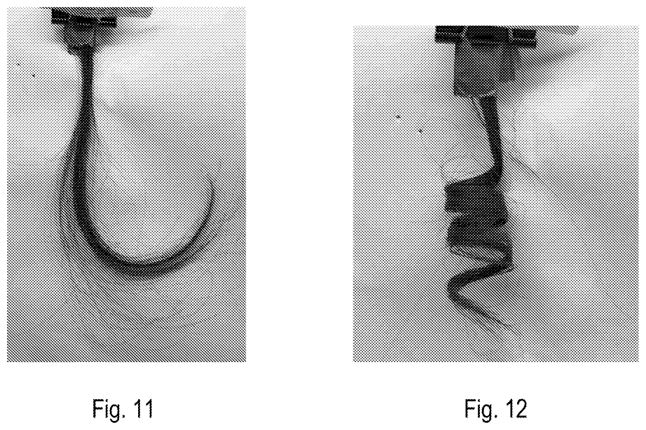

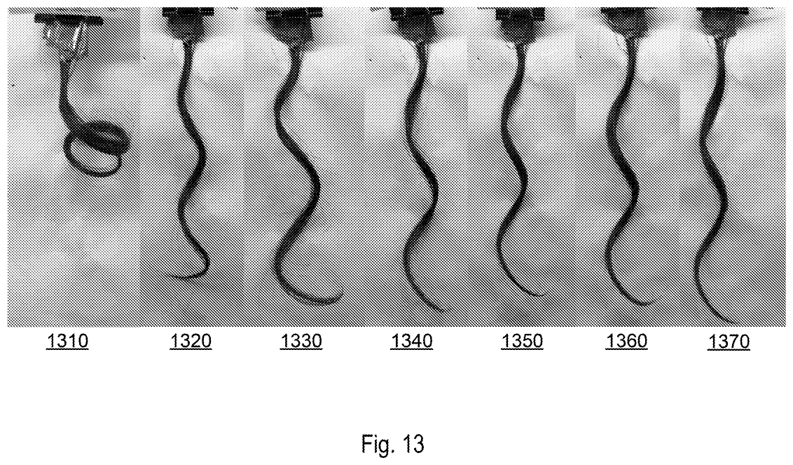

In Example 3, a solution comprising a photosensitizer and exogenous fiber was used to perform a cosmetic treatment on a subject's hair. FIG. 11 shows an image of the subject's hair prior to the cosmetic treatment. In the example, a phosphate-buffered saline (PBS) solution having a concentration of 8% (w/w) Keratin and 0.1% (w/w) Rose Bengal was applied to a subject's hair, and the hair was physically altered from its original shape into a curled state without the application of heat or other chemicals. The hair was then irradiated with electromagnetic radiation for a duration. FIG. 12 shows an image of the subject's hair after treatment, and FIG. 13 shows a series of images that illustrate the subject's hair after one wash 1310, after two washes 1320, after three washes 1330, after four washes 1340, after five washes 1350, after six washes 1360, and after eight washes 1370. The washing protocol included shampooing the hair, and washing with water.

The present invention has been described in terms of one or more preferred embodiments, and it should be appreciated that many equivalents, alternatives, variations, and modifications, aside from those expressly stated, are possible and within the scope of the invention.

1. A method of performing a cosmetic treatment, the method comprising:
(a) applying a photosensitizer to a keratin fiber of a subject while the keratin fiber is in a first physical state; and
(b) subjecting the keratin fiber and the photosensitizer to electromagnetic radiation selected to effectuate photocrosslinking or photoactivation on the keratin fiber to cause the keratin fiber to transition to a second physical state that is different than the first physical state.

2. The method of claim 1, wherein the electromagnetic radiation is applied at a wavelength between 400 and 700 nm.

3. The method of claim 1, wherein the electromagnetic radiation is applied at a power output between 60 and 120 Joules/cm².

4. The method of claim 1, wherein the photosensitizer is selected from Rose Bengal, riboflavin, and mixtures thereof.

5. The method of claim 1, wherein the keratin fiber is positioned within a strand of hair of the subject.

6. The method of claim 5, wherein step (b) further comprises subjecting the keratin fiber to physical forces a cosmetic device to arrange the keratin fiber in the second physical state.

7. The method of claim 6, wherein the cosmetic device is includes at least one of a curling iron, a hair straightener, a blow dryer, and a hair brush.

8. The method of claim 5 further comprising:
(a) applying a fibrous protein to the strand of hair on the subject;
(b) subjecting the fibrous protein, the photosensitizer, and the strand of hair to electromagnetic radiation selected to effectuate photocrosslinking between the fibrous protein and the keratin fiber within the strand of hair; and
wherein the photocrosslinking transitions the keratin fiber from the first physical state to the second physical state.

9. The method of claim 8, wherein the fibrous protein comprises keratin, collagen, synthetic fibers, artificial hair, hair, and mixtures thereof.

10. The method of claim 9, wherein the fibrous protein further comprises a therapeutic material coupled to the fibrous protein.

11. The method of claim 8, wherein the fibrous protein is positioned within an external strand of hair.

12. The method of claim 11, wherein the external strand of hair is photocrosslinked to the strand of hair on the subject.

13. The method of claim 8, wherein the second physical state of the keratin fiber has a mean diameter that is greater than the first physical state of the keratin fiber.

14. The method of claim 8, wherein the second physical state of the keratin fiber has a mass that is greater than the first physical state of the keratin fiber.

15. The method of claim 8, wherein the second physical state of the keratin fiber in the strand of hair comprises a color that is different than the first physical state.

16. The method of claim 1, wherein the first physical state comprises a wavy or curly configuration and the second physical state comprises a linear configuration.

17. The method of claim 1, wherein the first physical state comprises a linear configuration and the second physical state comprising a wavy or curly configuration.

18. The method of claim 1, wherein the keratin fiber is positioned within a nail of the subject.

19. The method of claim 18 wherein step (a) includes applying a fibrous protein to the nail on the subject and step (b) includes subjecting the fibrous protein, the photosensitizer, and the nail to the electromagnetic radiation.

20. The method of claim 19, wherein the fibrous protein comprises keratin, collagen, or mixtures thereof.

21. The method of claim 19, wherein the fibrous protein further comprises a therapeutic coupled to the fibrous protein.

22. The method of claim 21, wherein the therapeutic comprises an antibacterial, an antimicrobial, or mixtures thereof.

23. The method of claim 19, wherein the second physical state of the keratin fiber has a mass that is greater than the first physical state of the keratin fiber.

24. The method of claim 19, wherein the second physical state of the keratin fiber has a density that is greater than the first physical state of the keratin fiber.

25. A light-based system for treating and shaping a keratin fiber, the system comprising:
a fluid supply system comprising a vessel in fluid communication with a fluid outlet, wherein the vessel is configured to contain a photosensitizer and the fluid outlet is configured to dispense the photosensitizer at a target region that includes the keratin fiber;
an activation system configured with one or more light source, the light source configured to apply electromagnetic radiation at the target region; and
a processor having a memory and configured to be in electrical communication with the activation system, the processor being configured to implement stored instructions from the memory to cause the activation system to apply the electromagnetic radiation to the target region to effectuate photocrosslinking on the keratin fiber to cause a transition from a first physical state to a second physical state.

26. The light-based system of claim 25, wherein the processor is in electrical communication with a dispensing system to cause the dispensing system to dispense the photosensitizer to the target region to coat at least a portion of the keratin fiber with the photosensitizer.

27. The light-based system of claim 25 further comprising a cosmetic device configured to apply a physical force to the keratin fiber in the target region to move the keratin fiber from the first physical state to the second physical state.

28. The light-based system of claim 27, wherein the cosmetic device includes a heating element.

29. The light-based system of claim 27, wherein the cosmetic device includes a cooling element.

30. The light-based system of claim 25, wherein the cosmetic device comprises:
a pair of handles extending from a hinge, the pair of handles includes a first handle and a second handle;
a first conductive element attached on an inner surface of the first handle;
a second conductive element attached on an inner surface of the second handle;
at least one heating element in contact with the first conductive element and the second conductive element;
at least one fluid dispensing port configured to dispense the photosensitizer to the target region;
at least one light dispensing port configured to apply electromagnetic radiation to the target region; and
wherein the processor is in electrical communication with the at least one heating element and is programmed to cause the first conductive element and the second conductive element to increase in temperature.

31. The light-based system of claim 25, wherein the cosmetic device comprises:
a heat conducting body having an exterior surface and a hollow center;
at least one heating element disposed within the hollow center configured to heat the heat conducting body;
a handle coupled to the heat conducting body;
a clamp pivotally connected to the heat conducting body, the clamp being movable between an open position and a closed position, wherein the clamp is configured to rest on a surface of the heat conducting body when the clamp is in the closed position;
at least one fluid dispensing port configured to deliver the photosensitizer to the target region;
at least one light dispensing port configured to apply electromagnetic radiation at the target region; and
wherein the processor is in electrical communication with the at least one heating element to cause the at least one heating element to raise in temperature.

32. The light-based system of claim 25, wherein the cosmetic device comprises:
an elongated housing extending from an inlet to an outlet to define an airflow axis;
a fan positioned within the elongated housing to draw air into the elongated housing through the inlet and force air through the outlet toward a target region;
at least one heating element disposed within the housing;
at least one fluid dispensing port configured to dispense the photosensitizer to the target region;
at least one light dispensing port configured apply electromagnetic radiation at the target region;
the processor in electrical communication with the at least one heating element to cause the heating element to increase a temperature of the airflow through the airflow axis.

33. The light-based system of claim 25, wherein the cosmetic device comprises:
a handle extending from a first end to a second end;
at least one filament extending from an exterior surface of the handle, wherein the light source of the activation system is configured to cause the at least one filament to apply electromagnetic radiation to the target region.

34. The light-based system of claim 25, wherein the cosmetic device comprises:
a handle extending from a first end to a second end;
a porous media configured to the second end; and
at least one light dispensing port configured to apply electromagnetic radiation at the target region.

35. The light-based system of claim 25, wherein the cosmetic device comprises:
a container configured to receive the photosensitizer;
an applicator comprising a stem that extends between an applicator head and a handle, the handle configured to be removeably coupled to a top face of the container and the stem configured to be disposed within the container; and
at least one filament extending from an exterior surface of the applicator head, wherein the light source of the activation system is configured to cause the at least one filament to apply electromagnetic radiation to the target region.

36. The light-based system of claim 35, wherein the photosensitizer is suspended or dissolved in a gel.

37. The light-based system of claim 36, wherein the gel further comprises an exogenous fiber.

38. The light-based system of claim 37, wherein the photosensitizer is dissolved in a liquid solution.

39. The light-based system of claim 38, wherein the liquid solution further comprises an exogenous fiber.
